(19) United States
(12) Patent Application Publication
(10) Pub. No.: US 2005/0148881 A1
(43) Pub. Date: Jul. 7, 2005

(54) High-frequency intensity-modulated incoherent optical source for biomedical optical imaging

(76) Inventors: Ravel A. Fomitchov, Clifton Park, NY (US); Eugene Olczak, Glenville, NY (US); Stephen Lomnes, Albany, NY (US)

Correspondence Address: Dennis M. Flaherty, Esq.;Ostrager Chong & Flaherty LLP, 825 Third Avenue, 30th Floor, New York, NY, 10022-7519, US

(21) Appl. No.: 10/742,410

(22) Filed: Dec. 19, 2003

(51) Int. Cl. A61B 5/05

(52) U.S. Cl. 600/476

(57) ABSTRACT

An illumination device comprising: an incoherent light source; a non-imaging light collection system disposed to collect light emitted by the light source; an electro-optic modulator that receives light collected by the light collection system and modulates its intensity; and a light delivery system that receives intensity-modulated light output by the electro-optic modulator and illuminates an area with diverging intensity-modulated light. High-powered incoherent, multi-spectral illumination sources, such as lamps or LED arrays, are used. Non-imaging light collection elements, such as fiber optic bundles, total internal reflection lens arrays or compound parabolic light concentrators, are used to reduce the divergence of the light source for efficient transmission of light through an electro-optic modulator.

### BACKGROUND OF THE INVENTION

This invention generally relates to apparatus and methods for imaging using modulated light.

Biomedical optical imaging applications require a field of view that is near to 15 cm in diameter (178 cm²) and illumination intensity over that field of view in the range of 10-100 mW/cm². This implies the need for 1.78-17.8 W of optical power to be delivered to a viewed surface. High-frequency optical imaging techniques typically use coherent light sources such as lasers and laser diodes because of the ease with which their intensity can be modulated. However, the use of lasers with powers on the order of 17.8 W in a clinical diagnostic device is prohibitive because of regulatory and market acceptance concerns. Therefore high-frequency optical imaging methods currently employ lower-power lasers. However, the required illumination intensity limits the field of view of these devices to a few centimeters, which is not sufficient for large field of view clinical diagnostic imaging applications.

The trend in deep-tissue biomedical optical imaging is to increase the number of wavelengths of light in a single imaging setting. Typical coherent sources have a very narrow spectral bandwidth. High-frequency multi-spectral imaging is currently performed through the use of multiple channels of monochromatic coherent sources arranged in parallel. Each additional wavelength, or channel requires an additional optical source that greatly increases the complexity and cost of the instrumentation.

Alternatively, incoherent optical sources such as halogen lamps and arc lamps have been used for continuous-wave optical imaging applications, at the expense of the imaging sensitivity and the diagnostic information content provided by high-frequency optical imaging techniques.

There is a need for an optical source for use in biomedical imaging that provides optical safety, produces a large illumination area, and employs high-frequency modulation to prevent image degradation due to various instrumental or ambient noises or interferences. There is a further need for a technique and system that facilitates high-frequency optical imaging at different wavelengths using a single illumination source.

### BRIEF DESCRIPTION OF THE INVENTION

The foregoing needs are met by the method and apparatus disclosed herein, which employ high-frequency intensity modulation of an incoherent light source. In addition, the disclosed method and apparatus allow for the use of high-powered incoherent, multi-spectral illumination sources, such as lamps or LED arrays, in high-frequency optical imaging techniques. Non-imaging light collection elements, such as fiber optic bundles, total internal reflection (TIR) lens arrays or compound parabolic light concentrators, are used to reduce the divergence of the light source for efficient transmission of light through an electro-optic modulator whose transmissivity is modulated by applying a voltage across it. The method enables the use of inexpensive, safe, and wide-band illumination sources for large-area high-frequency fluorescence imaging of biological tissue for clinical applications. The main advantages of the use of incoherent light sources over coherent sources are: (1) decreased safety risk and regulation; (2) increased field of view; (3) decreased cost; and (4) ease of modification of the illumination wavelength.

One aspect of the invention is an illumination device comprising: an incoherent light source; a non-imaging light collection system disposed to collect light emitted by the light source; an electro-optic modulator that receives light collected by the light collection system and modulates its intensity; and a light delivery system that receives intensity-modulated light output by the electro-optic modulator and illuminates an area with diverging intensity-modulated light.

Another aspect of the invention is a method for illuminating a sample of biological tissue, comprising the following steps: (a) emitting incoherent light that diverges; (b) collecting a portion of the emitted incoherent light and outputting it with reduced divergence; (c) filtering the outputted incoherent light to pass selected wavelengths; (d) modulating the intensity of the filtered incoherent light; and (e) delivering the intensity-modulated incoherent light to a zone of illumination.

A further aspect of the invention is an illumination device comprising: a lamp; a bundle of optical fibers having input ends that surround a portion of the lamp to form a solid angle of light collection and also having outputs ends; an electro-optic modulator that receives light from the output ends of the optical fibers and modulates the intensity of that light; and a light delivery system that receives intensity-modulated light output by the electro-optic modulator and illuminates an area with diverging intensity-modulated light.

Yet another aspect of the invention is an illumination device comprising: an array of light-emitting diodes; an array of light-collecting elements disposed such that light from a respective light-emitting diode impinges on an input end of a respective light-collecting element; an electro-optic modulator that receives light from output ends of the light-collecting elements and modulates the intensity of that light; and a light delivery system that receives intensity-modulated light output by the electro-optic modulator and illuminates an area with diverging intensity-modulated light.

Other aspects of the invention are disclosed and claimed below.

### BRIEF DESCRIPTION OF THE DRAWINGS

FIG. 1 is a diagram showing the functionality of an incoherent optical source in accordance with the broad concept of the invention. The bullets indicate various non-limitative implementations for each component of the broadly conceived system.

FIG. 2 is a diagram showing light collection from an LED array using an optical fiber bundle in accordance with one embodiment of the first two stages depicted in FIG. 1.

FIG. 3 is a diagram showing light collection from an LED array using a total internal reflection (TIR) lens array in accordance with another embodiment of the first two stages depicted in FIG. 1.

FIG. 4 is a diagram showing light collection from an LED array using a compound parabolic concentrator array in accordance with a further embodiment of the first two stages depicted in FIG. 1.

FIG. 5 is a diagram showing light collection from a lamp using an optical fiber bundle in accordance with yet another embodiment of the first two stages depicted in FIG. 1.

FIG. 6 is a diagram showing one embodiment of the intermediate stages the functional components in accordance with one embodiment of certain intermediate stages depicted in FIG. 1.

FIG. 7 is a diagram showing light delivery from an electro-optic intensity modulator to biological tissue using an optical fiber bundle in accordance with one embodiment of the last stage depicted in FIG. 1.

FIG. 8 is a diagram showing light delivery from an electro-optic intensity modulator to biological tissue using beamforming optics in accordance with another embodiment of the last stage depicted in FIG. 1.

Reference will now be made to the drawings in which similar elements in different drawings bear the same reference numerals.

### DETAILED DESCRIPTION OF THE INVENTION

The invention is directed to providing safer, high-powered, incoherent light sources for use in optical imaging. Incoherent light sources are divergent sources that have a wide angular radiation pattern and are typically large illumination sources. Intensity modulation of coherent sources at high speeds can be done with electro-optic modulators. These modulators, however, are long crystals that have small cross-sectional areas, thus making it impossible to focus light using traditional optical components at a reasonable focal distance from an incoherent source without large losses. The invention employs non-imaging optics such as total internal reflection (TIR) lenses [see, e.g., U.S. Pat. No. 5,404,869], compound parabolic concentrators, and fiber optic couplers in conjunction with these divergent incoherent light sources to substantially reduce the effective divergence of the light. This substantially improves the transmission efficiency through the electro-optic modulator. The term “non-imaging” as used herein means that the device does not form an image of the light source. See, e.g., The Optics of Nonimaging Concentrators, W. T. Welford and R. Winston, Academic Press (1978).

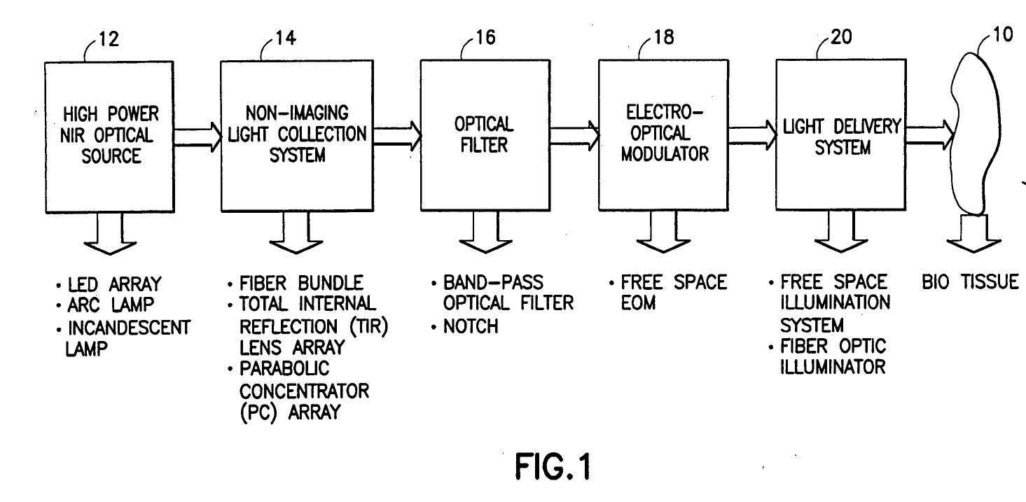

FIG. 1 is a diagram showing the functionality of a high-frequency intensity-modulated incoherent optical source for imaging a sample 10 of biological tissue. Near-infrared light is generated by a high-power incoherent optical source 12. That incoherent optical source may comprise a single or a plurality of light-emitting diodes (LEDs), arc lamps, incandescent lamps or equivalent devices. More specifically, the arc lamp may be a xenon arc lamp; the incandescent lamp may be a quartz tungsten halogen lamp. The arc lamp and the incandescent lamp are incoherent light sources, because they have very wide spectrum in the range of ˜200-2000 nm. The LEDs and LED arrays have an optical bandwidth on the order of ˜100 nm and also can be considered as incoherent sources because this bandwidth is approximately three orders of magnitude wider than a bandwidth of a typical coherent source such as a laser or laser diode.

The light from source 12 is concentrated using a non-imaging light collection system 14 comprising a multiplicity of beamforming elements. The multiplicity of beamforming elements may comprise a bundle of optical fibers, an array of total internal reflection (TIR) lenses, an array of compound parabolic concentrators, or equivalent devices. The light collected by light collection system 14 then passes through an optical filter 16, which may comprise a bandpass filter, a notch filter, a filter that passes long wavelengths only, or a filter that passes short wavelengths only. The wavelength that is passed through can be selected by optically filtering the undesired wavelengths out of the imaging chain. The filtered light then enters a free-space electro-optic modulator 18, which modulates the intensity of the light. As used herein, the term “free-space” means that the light does not travel through a light channel such as an optical fiber, but instead passes through air. The intensity-modulated light is then delivered to the sample 10 via a light delivery system 20, which may comprise a bundle of optical fibers or a free-space illumination system comprising beamforming optics. The term “optics” includes lenses, mirrors and other optical devices.

The above-described system enables the use of a single broadband optical source for producing a continuous, or discrete, range of wavelengths selected by optical filtering during high-frequency optical imaging. For example, the desired wavelength may be selected by changing the optical filter 16. This allows careful tuning of the illumination wavelength over a continuous range.

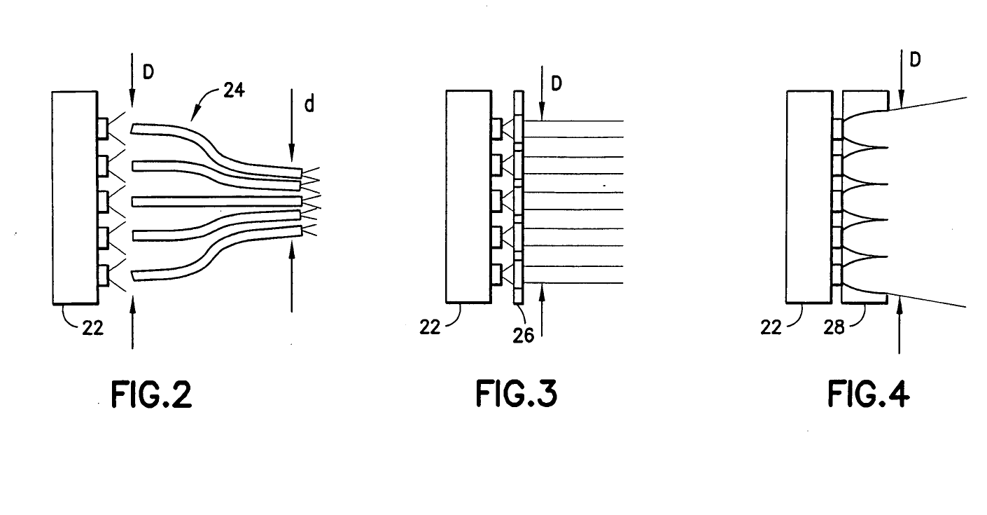

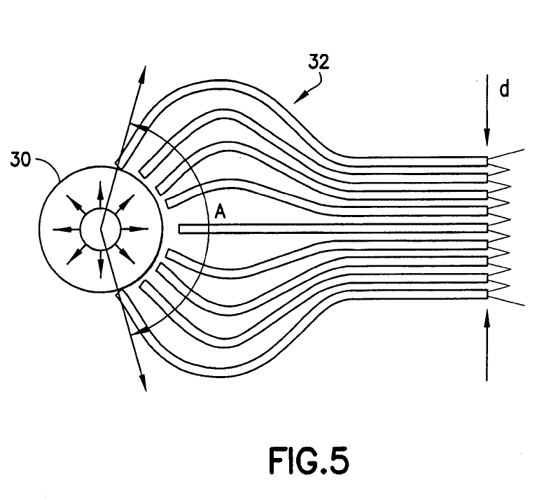

FIG. 2 shows one embodiment of the first two stages of the incoherent light source depicted in FIG. 1. The optical source comprises an LED array 22, while the light collection system comprises a bundle of optical fibers 24. Although only one dimension of the LED array is seen in FIG. 2, it should be appreciated that the LED array is two-dimensional with, e.g., LEDs arranged in rows and columns. (This is also true in FIGS. 3 and 4.) One end of each optical fiber is aligned with a respective LED of the array 22, while the other ends of the optical fibers are arranged closer together. The optical fibers are parallel at the output end of the bundle. As a result, the size D of the optical source is reduced to the diameter d of the optical fiber bundle 24. This arrangement reduces the divergence of the optical source to equal the numerical aperture of the fiber bundle.

FIG. 3 shows another embodiment of the first two stages of the incoherent light source in which the optical source comprises an LED array 22 and the light collection system comprises a TIR lens array 26. Each TIR lens is placed in front of a respective LED. As a result, the size D of the optical source stays the same. The divergence of the optical source is reduced due to the action of the individual TIR lenses.

FIG. 4 shows a further embodiment of the first two stages of the incoherent light source in which the optical source comprises an LED array 22 and the light collection system comprises an array 28 of compound parabolic concentrators. Each compound parabolic concentrator is optically coupled to a respective LED. As a result, the size D of the optical source stays the same. The divergence of the optical source is reduced due to the action of the individual TIR lenses compound parabolic concentrators.

FIG. 5 shows yet another embodiment of the first two stages of the incoherent light source depicted in FIG. 1. In this case, the optical source comprises a lamp 30, while the light collection system comprises a bundle of optical fibers 32. The optical fiber bundle is assembled from fibers having a low numerical aperture, e.g., on the order of 0.1. The input ends of the optical fibers surround a portion of the lamp to increase a solid angle of light collection. The input end of each optical fiber is aligned radially with the source of illumination. The angle of light collection is indicated by the arc A in FIG. 5. The optical fibers are close together and parallel at the output end of the bundle. As a result, the size D of the optical source is transformed to the diameter d of the optical fiber bundle 32. This arrangement reduces the divergence of the optical source to equal the numerical aperture of the fiber bundle.

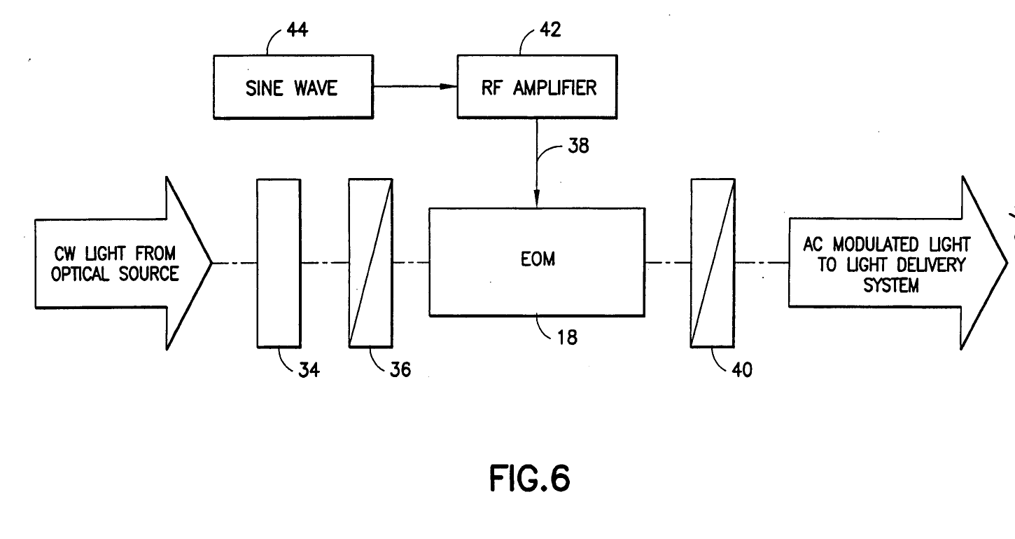

FIG. 6 shows one embodiment of those portions of the incoherent optical source disposed between the light collection and light delivery systems depicted in FIG. 1. The wide arrow on the left-hand side of the drawing represents continuous wave (CW) light from the light collection system. The wide arrow on the right-hand side of the drawing represents AC-modulated light being passed to the light delivery system. In accordance with this embodiment, the incoming CW light is filtered by an optical bandpass filter 34, which passes only the desired range of wavelengths. The filtered light is then passed through a polarizer 36 that polarizes the light. The polarized light then enters the free-space electro-optic modulator (EOM) 18. The EOM is made up of long crystals that have small cross-sectional areas. The reduced divergence of the light from the divergent incoherent light source, due to the effect of the light collection system, makes it possible to focus incoherent light using traditional optical components at a reasonable focal distance from the incoherent source without large losses. This substantially improves the transmission efficiency (i.e., optical coupling efficiency) through the electro-optic modulator 18.

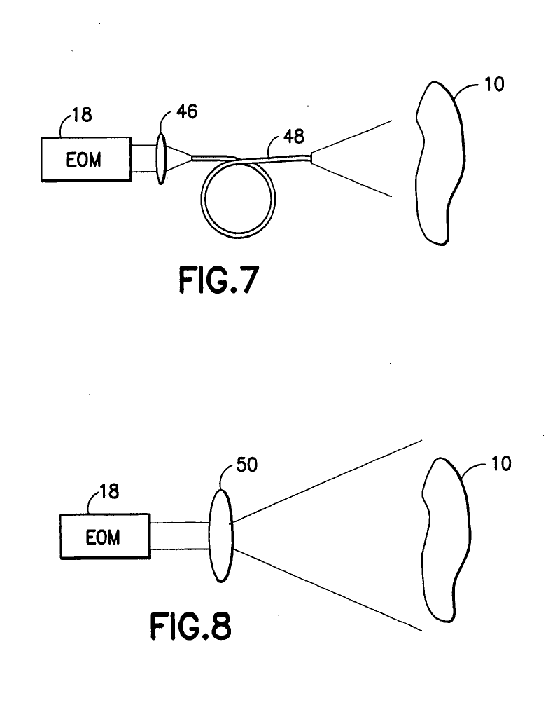

The intensity of the polarized optical signal is modulated by the electro-optic modulator 18 by changing the control voltage 38. The control voltage is output by a radiofrequency (RF) amplifier 42, which receives a sine wave signal at one of its inputs, the latter being generated by a sine wave generator 44. The intensity-modulated light then enters an analyzer 40, which transmits only plane-polarized light. The plane-polarized intensity-modulated light is then output to the light delivery system, embodiments of which are generally depicted in FIGS. 7 and 8.

FIG. 7 shows one embodiment of a light delivery system comprising beamforming optics 4 and an optical fiber bundle 48. For example, the beamforming optics 4 may comprise a lens that focuses the intensity-modulated light from the EOM 18, directing the focused beam toward the input end of the bundle 48. The intensity-modulated light diverges as it exits the output end of the bundle 48, which is directed toward the sample 10 being imaged.

Alternatively, the light delivery system may comprise beamforming optics 50 (e.g., a lens, mirrors, etc.) without an optical fiber bundle. The optics 50 forms a diverging beam that illuminates the sample 10.

The method and apparatus described above allows one to increase overall light intensity on a sample without major safety measures. The high optical power of the source also increases the field of view of an imager by providing a larger illumination area. Moreover, because of the linearity of the modulation, the modulation frequency is not limited by the parameters of the optical source (such as the rise and fall times of LEDs). The invention also enables changing the wavelength of the transmitted light (i.e., by changing the filter) without changing the illumination source.

While the invention has been described with reference to preferred embodiments, it will be understood by those skilled in the art that various changes may be made and equivalents may be substituted for elements thereof without departing from the scope of the invention. In addition, many modifications may be made to adapt a particular situation to the teachings of the invention without departing from the essential scope thereof. Therefore it is intended that the invention not be limited to the particular embodiment disclosed as the best mode contemplated for carrying out this invention, but that the invention will include all embodiments falling within the scope of the appended claims.

1. An illumination device comprising:
a incoherent light source;
a non-imaging light collection system disposed to collect light emitted by said light source;
an electro-optic modulator that receives light collected by said light collection system and modulates its intensity; and
a light delivery system that receives intensity-modulated light output by said electro-optic modulator and illuminates an area with diverging intensity-modulated light.

2. The device as recited in claim 1, further comprising an optical filter that the collected light passes through before reaching said electro-optic modulator.

3. The device as recited in claim 1, further comprising a polarizer that the collected light passes through before reaching said electro-optic modulator.

4. The device as recited in claim 3, further comprising an analyzer that the intensity-modulated light passes through before reaching said light delivery system.

5. The device as recited in claim 1, wherein said light source comprises an array of light-emitting diodes.

6. The device as recited in claim 1, wherein said light source comprises an arc lamp.

7. The device as recited in claim 1, wherein said light source comprises an incandescent lamp.

8. The device as recited in claim 1, wherein said light collection system comprises a bundle of optical fibers.

9. The device as recited in claim 8, wherein said optical fibers of said bundle comprise input ends arranged to occupy a first area and output ends arranged to occupy a second area less than said first area, said output end being substantially mutually parallel.

10. The device as recited in claim 1, wherein said light collection system comprises an array of total internal reflection lenses.

11. The device as recited in claim 1, wherein said light collection system comprises an array of compound parabolic concentrators

12. The device as recited in claim 1, wherein said light delivery system comprises a bundle of optical fibers.

13. The device as recited in claim 1, wherein said light delivery system comprises beamforming optics.

14. The device as recited in claim 1, wherein said light source comprises a lamp and said light collection system comprises a bundle of optical fibers having input ends that surround a portion of said lamp to form a solid angle of light collection.

15. The device as recited in claim 14, wherein the input end of each optical fiber is aligned radially with a source of illumination inside said lamp.

16. A method for illuminating a sample of biological tissue, comprising the following steps:
(a) emitting incoherent light that diverges;
(b) collecting a portion of the emitted incoherent light and outputting it with reduced divergence;
(c) filtering the outputted incoherent light to pass selected wavelengths;
(d) modulating the intensity of the filtered incoherent light; and
(e) delivering the intensity-modulated incoherent light to a zone of illumination.

17. The method as recited in claim 16, further comprising the step of polarizing the filtered incoherent light before step (d).

18. The method as recited in claim 17, further comprising the step of analyzing the intensity-modulated incoherent light before step (e).

19. An illumination device comprising:
a lamp;
a bundle of optical fibers having input ends that surround a portion of said lamp to form a solid angle of light collection and also having outputs ends;
an electro-optic modulator that receives light from said output ends of said optical fibers and modulates the intensity of that light; and
a light delivery system that receives intensity-modulated light output by said electro-optic modulator and illuminates an area with diverging intensity-modulated light.

20. The device as recited in claim 19, wherein the input end of each optical fiber is aligned radially with a source of illumination inside said lamp.

21. The device as recited in claim 19, wherein said lamp comprises an arc lamp.

22. The device as recited in claim 19, wherein said lamp comprises an incandescent lamp.

23. The device as recited in claim 19, further comprising an optical filter that the light emitted from the output ends of said optical fibers passes through before reaching said electro-optic modulator.

24. An illumination device comprising:
an array of light-emitting diodes;
an array of light-collecting elements disposed such that light from a respective light-emitting diode impinges on an input end of a respective light-collecting element;
an electro-optic modulator that receives light from output ends of said light-collecting elements and modulates the intensity of that light; and
a light delivery system that receives intensity-modulated light output by said electro-optic modulator and illuminates an area with diverging intensity-modulated light.

25. The device as recited in claim 24, wherein each of said light-collecting elements comprises a respective optical fiber.

26. The device as recited in claim 24, wherein each of said light-collecting elements comprises a respective total internal reflection lens.

27. The device as recited in claim 24, wherein each of said light-collecting elements comprises a respective compound parabolic concentrator.
